(19) United States
(12) Patent Application Publication
(10) Pub. No.: US 2012/0231180 A1
(43) Pub. Date: Sep. 13, 2012

(54) PROCESS OF PURIFYING RUTHENIUM PRECURSORS

(75) Inventors: Bin XIA, Plano, TX (US); Zhiwen Wan, Plano, TX (US); Ashutosh Misra, Plano, TX (US); Olivier Letessier, San Jose, CA (US)

(73) Assignee: Air Liquide Electronics U.S. LP, Dallas, TX (US)

(21) Appl. No.: 13/230,033

(22) Filed: Sep. 12, 2011

Related U.S. Application Data
(63) Continuation of application No. 12/437,224, filed on May 7, 2009, now abandoned.
(60) Provisional application No. 61/051,561, filed on May 8, 2008.

(51) Int. Cl. C09D 5/00; C23C 16/50; C23C 16/06

(52) U.S. Cl. 427/569; 106/287.18; 427/255.28

(57) ABSTRACT

Disclosed are methods of purifying a ruthenium containing precursor by removing oxygen from the ruthenium containing precursor by flowing an inert gas through the ruthenium containing precursor. Also disclosed are methods of forming an improved ruthenium containing film using the purified ruthenium containing precursor.

### CROSS REFERENCE TO RELATED APPLICATION

The present application is a continuation of U.S. patent application Ser. No. 12/437,224, filed May 7, 2009, which claims the benefit of U.S. Provisional Application Ser. No. 61/051,561, filed May 8, 2008, herein incorporated by reference in its entirety for all purposes.

### FIELD OF THE INVENTION

The present invention relates to a process for purifying ruthenium precursors, particularly ruthenium precursors to be used in semiconductor manufacturing processes.

### BACKGROUND OF THE INVENTION

Ruthenium is a precious metal with high conductivity, high oxidation resistance and temperature stability. Ruthenium containing films that include ruthenium or ruthenium oxide can be used in many applications such as semiconductor fabrication processes and magnetic recording applications. In addition, ruthenium is a promising material for gate metal in CMOS transistors that are used with high-k dielectric materials, capacitor electrodes with tantalum pentoxide or BST perovskite materials in memory applications such as DRAM and copper barriers and magnetic recording applications. For example, ruthenium could replace currently used tantalum nitride as a copper diffusion barrier and could simplify the manufacturing process in the technology node 45 nm or beyond. Ruthenium films can be deposited using CVD, ALD or PVD to form a thin layer to separate low-k IMD and copper interconnect in the CMOS transistor, thereby eliminating the need to form complicated Ta/TaN/Cu seed barrier layer in the present technology. Ruthenium films can also be etched and patterned using O₂ plasma or fluorine-based plasma.

A variety of precursors have been used to deposit ruthenium containing films by CVD or ALD. The precursor utilized often depends upon the process utilized and is typically chosen on the basis of precursor volatility, delivery of the precursor, reactivity, thermal stability, film composition, film purity (absence of impurities), film performance and so forth. The most common precursors utilized are Ru(C₅H₅)₂ (bis(cyclopentadienyl)ruthenium), Ru₃(CO)₁₂ (dodecacarbonyl triruthenium), or their derivatives such as ethyl Ru(Et-C₅H₄)₂ or (C₅H₄)Ru(CO)₃. Note that all of these precursors contain carbon.

Organometallic ruthenium precursors which have direct Ru—C bonds require one or more oxidizing agents such as O₂, O₃, N₂O, NO, NO₂, or H₂O₂ to remove the organic ligands and to form ruthenium films. However, these oxidizing agents have the effect of oxidizing the substrate. Oxides that are formed may increase the resistivity of the ruthenium film and deteriorate performance. In the case of insufficient oxidation, carbon can incorporate into the films and lower the performance. In the case of over-oxidation, RuOₓ will form thereby resulting in the need for post-CVD processing, such as annealing in H₂, to reduce the RuOₓ.

The resistivity of deposited ruthenium films is a key feature in determining the ruthenium performance. Pure ruthenium has a resistivity of 7 ohm·m, while the resistivity of CVD/ALD deposited films is higher because the films contain impurities such as carbon, oxygen or hydrogen.

Another ruthenium compound, ruthenium tetraoxide, however, is a good precursor to form ruthenium containing films by CVD or ALD since ruthenium tetraoxide does not contain any carbon or hydrogen and is easy to be reduced to ruthenium without oxygen incorporation. As a result, conformal films with a thickness from a few angstroms to thousands of angstroms can be readily controlled during deposition on a wafer such as silicon or aluminum oxide.

However, ruthenium tetraoxide is temperature and light sensitive and is only fairly stable at room temperature and ambient pressure. Pure ruthenium tetraoxide is difficult to handle due to the risk of explosion resulting from self decomposition at elevated temperatures such as about 130° C. In addition, ruthenium tetraoxide is a solid at room temperature and therefore it is not easy to control constant delivery to a reaction chamber where a uniform ruthenium film will be formed. For these reasons, fluorinated solvents are used to dissolve ruthenium tetraoxide. The resulting solution can be either bubbled through by a gas or evaporated in a vaporizer to deliver ruthenium tetraoxide vapor to a reaction chamber in order to form ruthenium containing films.

The ruthenium precursor that will be used to form the film can be synthesized by extracting ruthenium tetraoxide [the ruthenium compound] from an aqueous solution to an organic solvent. Ruthenium tetraoxide is formed in-situ in an aqueous solution by mixing a ruthenium containing compound as a starting chemical with at least an oxidizer that can dissolve in water.

Some examples of ruthenium starting compounds for preparing ruthenium tetraoxide include, but are not limited to, ruthenium dioxide, ruthenium chloride, ruthenium powder, or ruthenium nitrosyl. In other instances, commercially available ruthenium tetraoxide aqueous solutions can be used. The oxidizer used can be selected from sodium periodate, cerium ammonium nitrate, perchloric acid, ammonium persulfate, periodic acid, ozone water, and the like.

By mixing a ruthenium starting compound with an oxidizer, ruthenium tetraoxide can be formed and dissolved in an aqueous solution. The resulting aqueous solution is clear, yellow and has an acute odor. Next, the resulting aqueous solution is mixed with an organic solvent, preferably a fluorinated solvent, in order to extract ruthenium tetraoxide from the aqueous solution into the organic solvent. The solution stability depends upon the type of oxidizers, ruthenium starting compound, solvent, and synthesis conditions.

After extraction, the organic solvent includes the ruthenium tetraoxide. The raw product, i.e. the organic phase, is then separated from the aqueous solution by a separation process, for example, a separation funnel. The product is ready for use at this point. However, due to dissolved moisture and possibly other impurities in the raw product, it is desirable to remove these impurities from the ruthenium precursor in order to obtain a highly purified ruthenium precursor. Failure to remove the impurities can cause a variety of problems. For example: the dissolved moisture in the raw product could significantly affect the film deposition process; synthesis additives in the aqueous solution may be carried into the organic solution, and ultimately to the process chamber, which may result in unwanted reactions; the impurities could have adverse effects on the film deposition process such as high electrical resistance due to formation of ruthenium oxide or existence of impurities, thickness non-uniformity, etc and the moisture could also affect stability of the product.

Therefore, it is desirable to have highly purified precursors in order to achieve high quality films. Accordingly, there is a need for an easy and efficient process to remove impurities from ruthenium precursors prior to the precursors being used to form ruthenium films.

### SUMMARY OF THE INVENTION

It has now been found that it is possible to remove a large portion of the impurities that are present in ruthenium precursors that will be used to produce ruthenium films. As a result, the ruthenium films prepared using the purified ruthenium precursor are of a higher quality then those films prepared using ruthenium precursors that have not been purified using the processes of the present invention. The present invention provides for a process that purifies ruthenium precursors by removing impurities from the ruthenium precursor. The process of the present invention involves contacting the ruthenium precursor with one or more drying agents for a period of time followed by separating the one or more drying agents from the ruthenium precursor to achieve a final product that is a purified ruthenium precursor. In an alternative embodiment, the process comprises passing the ruthenium precursor through a column that contains one or more drying agents followed by a filtration step to remove any residual material. Purified ruthenium precursors produced using the processes of the present invention, when utilized to make ruthenium containing films for semiconductor use, result in higher quality films.

### BRIEF DESCRIPTION OF THE FIGURES

FIG. 1 provides a graph of oxygen level in-site monitoring as a function of an argon purge through the solvent used.

### DETAILED DESCRIPTION OF THE PRESENT INVENTION

The present invention provides for a process for removing impurities from a ruthenium precursor. With regard to the present invention, the phrases “ruthenium precursor” and “ruthenium precursors” refer to the ruthenium compound/solvent solution obtained when the ruthenium compound to be utilized is dissolved in a solvent for disposition on the substrate as discussed hereinbefore. Typically such precursors, due to the manner in which they are produced, contain a variety of impurities which can either directly or indirectly affect the quality of the film to be produced. One specific class of such ruthenium precursors are the ruthenium precursors disclosed in U.S. Patent Publication No. 2008/0214003, incorporated herein in its entirety by reference.

Furthermore, as used herein, the term “impurities” refers to a variety of byproducts that may be present in the ruthenium precursor mixture of ruthenium compound and solvent due to the manner in which the ruthenium precursor is produced or are present due to contamination. For purposes of the processes of the present invention, the term impurities is limited to those impurities to be removed from the ruthenium precursors that include moisture, as well as any impurity which is capable of being dissolved in water or organic solvent, particles and air (in the case where a reducing gas will be used in film deposition). By way of unlimited example, such impurities include moisture, cations, and anions. Moisture, being the most common impurity, is also the most damaging impurity and the removal of moisture is the main concern of the process of the present invention.

The processes of the present invention are particularly useful for the removal of impurities in ruthenium precursors which include ruthenium compounds that may be used to prepare films for semiconductors, magnetic recording devices, catalysts that contain ruthenium, and certain sensors and which must be dissolved in an inert organic solvent in order to be utilized (for example, deposited as a film on a substrate). More specifically, an example of such a compound includes, but is not limited to, ruthenium tetroxide (RuO₄).

With regard to the specifically noted ruthenium precursors, the inert organic solvent utilized to form these ruthenium precursor will typically be an organic solvent such as those also disclosed in U.S. Patent Publication No. 2008/0214003. More specifically, the organic solvents are those that are known for dissolving ruthenium compounds for the purpose of disposition of ruthenium films on substrates. Such organic solvents include, but are not limited to, non-flammable solvents such as fluorinated solvents. In other embodiments, two or more solvents will be utilized to form the ruthenium precursor. In those cases, the solvents can each be described according to the general formula: CₓH_{y}F_{z}OₜNᵤ, wherein x≧3; y+z≦2x+2; z≧1, t≧0, u≧0; and t+u≧0 and wherein x, y, z, t, and u are all integers. Several solvents which satisfy this general formula include, but are not limited to Methyl perfluoropropyl ether; methyl nonafluorobutyl ether; ethyl nonafluorbutyl ether; 1,1,1,2,2,3,4,5,5,5-decafluoro-3-methoxy-4-(trifluoromethyl)-Pentane; 3-ethoxy-1,1,1,2,3,4,4,5,5,6,6,6-dodecafluoro-2-trifluoromethyl-hexane; C₉F₁₂N; C₁₂F₂₇N; C₁₂F₃₃N; C₆F₁₄; C₈F₁₆; C₇F₁₆; C₅F₁₀H₂; C₄F₅H₅; 1,1,2,3,3 penta fluoro propane; CF₃CFHCF₂CH₂OCF₂CFHOC₃F₇; and C₃F₇OCFHCF₂CH(CH₃)OCF₂CFHOC₄F₉. In one embodiment of the present process, the solvent mixture used to form the ruthenium precursor is a mixture of methyl nonafluorobutyl ether and ethyl nonafluorbutyl ether. Both of these are available commercially from the 3M Company, and are sold under the trade names of Novec HFE 7100 and Novec HFE 7200. C₅F₁₀H₂ is also commercially available from DuPont under the trade name of Vertrel.

In one embodiment of the process of the present invention, the ruthenium precursor is contacted with one or more drying agents in order to remove impurities from the ruthenium precursor. The key is to choose a drying agent that has a strong drying capacity, that has a strong attraction to moisture and that is also inert (does not react with chemicals). As used herein with regard to the embodiments of the present invention, the phrase “drying agents” refers to the use of one or more materials which are capable of removing moisture and materials that are capable of being dissolved in water from a ruthenium precursor. The one limiting factor with regard to the drying agents utilized in the present processes is that the drying agents cannot be materials that would react with the ruthenium compound or the organic solvent. More specifically, the drying agent utilized should not contain reducing agents such as lithium aluminum hydrides, magnesium, sodium, etc. as such products would destroy the ruthenium precursor and would likely present safety issues. Non-limiting examples of the drying agents that may be used in the present processes include molecular sieves, alumina, silica gels, calcium sulfate, calcium chloride, Drierite, sodium sulfate, magnesium sulfate and like materials. The phrase “like materials” refers to additional materials (1) which are considered drying agents in that they function to “dry” the ruthenium precursor by removing the impurities in the same manner as achieved through the use of molecular sieves, aluminas, silica gels, calcium sulfate, calcium chloride, drierite and sodium sulfate and (2) which do not react with the ruthenium compound or the organic solvent. Of the drying agents noted, the most preferred are molecular sieves, alumina and silica gels. Of these preferred drying agents, the most preferred is molecular sieves.

When the one or more drying agents utilized are molecular sieve, non-limiting examples of the type of molecular sieves that can be used include, but are not limited to, molecular sieves which are synthesized or which are commercially available molecular sieves such as 3 A molecular sieves, 4 A molecular sieves, 5 A molecular sieves, 10× molecular sieves or 13× molecular sieves. The molecular sieve may be in a variety of forms and sizes, including as a powder or as pellets or beads and extrudated strips. Such pellets or beads are available in a large variety of sizes, the size utilized being dependent upon a variety of factors, including but not limited to the size of the bed in which the zeolite will be located and the amount of ruthenium precursor to be purified. For example, the beads utilized can range in size from about 1/16 inch (0.16 cm) to about ½ inch (1.3 cm), even more preferably from about ⅛ inch (0.3 cm) to about ¼ inch (0.6 cm), in diameter. Such molecular sieves are readily know to those of ordinary skill in the art and may be obtained from a variety of commercial sources. Prior to use, the molecular sieve utilized should be dried/activated. This is typically done by heating the molecular sieve in an over or microwave to a certain temperature for a certain period of time. Those of ordinary skill in the art will recognize that in many instances the manufacturer will provide directions on how to dry/active the particular molecular sieve in order to assure maximum drying. In addition, with regard to the molecular sieves, those of ordinary skill in the art will also recognize that at some point the molecular sieves will become loaded and therefore will not continue to function at a high efficiency (resulting in less efficient removal of the impurities). Accordingly, the molecular sieves will have to be monitored and removed once they are close to being fully loaded. The molecular sieves can be regenerated and reused or simply replaced with new molecular sieves. Accordingly, the process of the present embodiment is preferably conducted batchwise with regeneration of the molecular sieve or replacement of the molecular sieve between batches.

When alumina is used, non-limiting examples of the type of alumna that may be used include, but are not limited to, aluminum oxides (including hydrated) and their various forms. Those of ordinary skill in the art will recognize that the issue of regeneration or replacement of the drying agents is applicable to all drying agents. Accordingly, any of the methods known in the art for regenerating drying agents may be utilized or the drying agents may simply be replaced on a regular basis.

The ruthenium precursor is contacted with one or more drying agents in order to remove impurities from the ruthenium precursor. As by definition the impurities to be removed include moisture, the contact between the ruthenium precursor and one or more drying agents must take place in an inert atmosphere—under a blanket of inert gas. In other words, the process must be carried out dry—without the presence of moisture. The inert gas utilized can be any gas which does not react with the ruthenium precursor (with the ruthenium compound or the organic solvent). Therefore, the inert gas may be selected from dry air, dry oxygen, nitrogen, argon, carbon dioxide and helium.

The contact between the ruthenium precursor and the one or more drying agents may be carried out in two different manners (two different embodiments). The first embodiment involves contacting the solvent based ruthenium precursor with the one or more drying agents by initially mixing the ruthenium precursor and the one or more drying agents and then allowing the mixture to remain stationary (allowing the ruthenium precursor and one or more drying agents to stay in contact with one another) for a period of time sufficient to allow for the impurities present in the ruthenium precursor to adsorb on to the one or more drying agents. The second step of the process involves separating the one or more drying agents which now have at least a portion of the impurities adsorbed thereto from the ruthenium precursor. The initial mixing may occur in a variety of ways. For example, the mixing may be carried out without any actual use of outside physical mixing (without the use of an agitator or a magnetic stir bar)—the initial mixing may simply occur by pouring the ruthenium precursor and one or more drying agents together into a flask. In this case, the ruthenium precursor may be poured in first followed by the one or more drying agents or the one or more drying agents may be poured in followed by the ruthenium precursor. In alternative embodiments though, it is possible to apply outside physical mixing such as for example by adding a magnetic stir bar to the flask where the ruthenium precursor and one or more drying agents are added or by placing the flask in which the ruthenium precursor and one or more drying agents are added into a device which will actually physically shake or agitate the flask. In still further embodiments, the two components may be poured together and then on occasion be agitated slightly to allow for increased contact (through the use of a stir bar, a shaker or any other means for producing physical agitation).

In this particular embodiment, the ruthenium precursor is allowed to stay in contact with the one or more drying agents for a period of time sufficient to allow for removal of at least 50% of the impurities present in the ruthenium precursor, preferably at least 70% of the impurities present and even more preferably at least 90% of the impurities present. Depending upon the ultimate use of the ruthenium precursor and the actual ruthenium compound and solvent being utilized, in some embodiments of the present invention, the objective of the process is to achieve a purified ruthenium precursor having less than 100 ppm impurities, even more preferably less than 50 ppm impurities and even more preferably, less than 20 ppm impurities. Those of ordinary skill in the art will recognize that the purer the ruthenium precursor, the better for any film that is to be deposited using this precursor.

The actual time during which the ruthenium precursor will be in contact with the one or more drying agents in this embodiment will vary widely depending upon a variety of factors including the volume to be treated (small batch versus large batch), the degree of impurities, the ruthenium precursor, the organic solvent utilized and the actual drying agents utilized. Typically, the length of time that the ruthenium precursor and one or more drying agents are in contact will range anywhere from about 10 minutes to about 24 hours, preferably from about one hour to about 12 hours. Accordingly, when the batch is small, the length of time will typically be in the lower time range (from about 10 minutes to about 4 hours) while when the batch is large, the length of time will be in the higher time range (from about 8 hours to about 24 hours). As used herein, the term “small” refers to bench scale batches that comprise from about fifty grams to a few hundred grams (from about 50 grams to about 400 grams) while the term “large” refers to commercial scale batches that comprise from greater than about 400 grams up to about 10 kilos.

The temperature at which contact in the process of the present embodiment is carried out is not necessarily critical to the process. Typically, the process will be carried out at about room temperature (25° C.) although higher and lower temperatures are contemplated to be within the scope of the present invention. The lower limit of the temperatures will be determined based on the freezing point of the actual organic solvent and ruthenium precursor utilized. In many instances, those of ordinary skill in the art will recognize that taking into account the freezing point of the organic solvent and ruthenium precursor that the lower limit will typically be no lower than about −20° C. With regard to the upper temperature limit for carrying out this embodiment of the process of the present invention, this limit is determined by the stability of the ruthenium precursor and the organic solvent. Accordingly, the upper limit will typically be at most about 80° C. As noted though, the preferred temperature will be room temperature (25° C.) plus or minus 10° C. (from about 15° C. to about 30° C.).

The process of the present invention is preferably carried out at ambient pressure although higher and lower pressures can be utilized. When the process is carried out at high pressure, the pressure will typically be no higher than about 250 psi. When the process is carried out at lower pressure, the pressure will typically be no lower than about 50 torr although in certain instances it may be as low as 10 torr.

After the period of time in which the ruthenium precursor and one or more drying agents are in contact has ended, the one or more drying agents are separated from the “dried” ruthenium precursor (the purified ruthenium precursor). This separation may occur through the utilization of a filter. The filter must be of the type that the material from which the filter is constructed will not react with the ruthenium precursor (the ruthenium compound or the organic solvent). Therefore, the filter utilized should be constructed out of Teflon, stainless steel, steel alloy any other type of material that will not react with the ruthenium precursor or the organic solvent. The pore size of the filter must be such as to allow for the passage of the purified ruthenium precursor while at the same time retaining the one or more drying agents that are loaded with the impurities removed from the ruthenium precursor. Typically the pore size will range anywhere from about 0.1 microns to about 20 microns, with the actual size utilized depending upon the ultimate application for the ruthenium precursor. The mixture of ruthenium precursor and one or more drying agents will be placed in the filter and allowed to filter either using gravity or using pressure. When pressure is used, the amount of pressure utilized will be dependent upon the type of drying agents utilized and the type of filter utilized. The pressure can be applied through the use of a filter that includes a flow pump. Such filter/flow pump combinations are readily known by those of ordinary skill in the art.

The separation step is carried out under the same conditions as the contacting step (both under a blanket of inert gas and at the same temperature and pressure). Once the purified, filtered ruthenium precursor is obtained, it is also stored under a blanket of inert gas until used.

With regard to this first embodiment, the ratio of drying agent (cumulative amount of drying agent) to ruthenium precursor will typically range from about 1:1 (for example 100 grams of drying agent per 100 grams of ruthenium precursor) to about 1:100 (for example, 1 gram of drying agent per 100 grams of ruthenium precursor), preferably from about 1:10 (for example 1 gram of drying agent per 10 grams of ruthenium precursor) to about 1:50 (for example, 1 gram of drying agent per 50 grams of ruthenium precursor).

The second manner of contacting the ruthenium precursor with the one or more drying agents comprises an embodiment which uses a dynamic flowing process. As used herein, the phrase “dynamic flowing process” refers to the passing of the ruthenium precursor as described hereinbefore with or without the assistance of pressure through a column that contains one or more drying agents as described hereinbefore. In the most preferred alternative of this embodiment, the one or more drying agents will be selected from molecular sieves as described hereinbefore, preferably in the form of beads or pellets. The actual size of the beads or pellets will be dependent upon the quantity of product that needs to be dried as well as the size of the column. Typically, the bead or pellet size will range from about 1/16 inch (0.16 cm) to about ½ inch (1.3 cm), preferably from about ⅛ inch (0.3 cm) to about ¼ inch (0.6 cm), in diameter. In this alternative embodiment, the one or more drying agents are placed in a column which has a particulate filter connected to the end of the column. While the type of column utilized is not critical to the process of the present invention what is critical is that the column be composed of a material that is inert (does not react with the ruthenium precursor—ruthenium compound and solvent). Preferably the column utilized is a coated or uncoated stainless steel, glass, quartz, alumina or other ceramics column. The ruthenium precursor is passed through the column. As the ruthenium precursor flows through the column, impurities in the ruthenium precursor are adsorbed onto the one or more drying agents positioned in the column. The passage of the ruthenium precursor may take place with the aid of gravity or with the aid of pressure or vacuum.

Once the ruthenium precursor passes through the column, it then passes into the filtration unit that is attached to the column. The filtration unit serves to remove residual particles that may be carried from the column with the ruthenium precursor as it passed through the column, the residual particles typically resulting from the drying agent or the synthesis of the raw product. As noted above, pressure may be applied to aid in the flow of the ruthenium precursor though not only the column but also through the filter. When pressure is used, the amount of pressure utilized will be such that the driving force for the ruthenium precursor is greater than the flow through the column (in order to ensure that the column does not back up or that flow through the column does not stop) taking into consideration the type of drying agents employed as well as the physical characteristics of the drying agents. The pressure can be applied through the use of a filter that includes a flow pump. Such filter/flow pump combinations are readily known by those of ordinary skill in the art.

The contact (drying)/filtration steps may optionally be repeated one or more times depending upon the solvent utilized, the amount of impurities present in the ruthenium precursor and the filtration efficiency (the drying agents utilized). While it is difficult to obtain an exact measure of the impurities present in the ruthenium precursor, a good indication of the degree of impurities present and accordingly the number of passes through the column that are necessary to remove the impurities may be obtained by measuring the degree of impurities in the organic solvent to be used. In order to do this, the initial impurities present in the organic solvent (especially the moisture present) are measured. Once this baseline is established, the organic solvent is passed through the column/filter configuration that contains the drying agents that are to be used for drying the ruthenium precursor. The amount of moisture present in the organic solvent is measured after each pass thereby giving an indication of the amount of moisture removed in each pass. By comparing the moisture level obtained after each pass through the column/filter configuration, it is possible to determine how many times the actual ruthenium precursor (ruthenium compound in organic solvent) should be passed through the column/filter configuration. Note that the type of filter used as a part of the filtration unit is the same as that described hereinbefore with regard to the first embodiment.

By way of example, the table below provides a determination of the degree of moisture present (in ppm's) for a mixture of methyl nonafluorobutyl ether and ethyl nonafluorobutyl ether solvent prior to being passed through a column containing 4 A molecular sieve and after a variety of passes through a column.

Number of passes thru
drying column

| | 0 | 1 | 2 | 3 | 4 | 5 |
|---|---|---|---|---|---|---|
| Moisture (ppm) | 22.4 | 0.2 | 0.3 | 0.1 | 0.3 | 0.3 |

After the above purification, film deposition processes are significantly improved as film uniformity is improved and batch-to-batch deposition performance is more repeatable and consistent. Without purification, film resistance from the raw product can go up to 500 ohm/square. After purification, the film resistance drops to around 15 ohm/square.

As in the first embodiment, the process of this particular embodiment seeks to allow for removal of at least 50% of the impurities present in the ruthenium precursor, preferably at least 70% of the impurities present and even more preferably at least 90% of the impurities present. Depending upon the ultimate use of the ruthenium precursor and the actual ruthenium compound and solvent being utilized, in some embodiments of the present invention, the objective of the process is to achieve a purified ruthenium precursor having less than 100 ppm impurities, even more preferably less than 50 ppm impurities and even more preferably, less than 20 ppm impurities.

Also as in the first embodiment, the ratio of drying agent (cumulative amount of drying agent) to ruthenium precursor will typically range from about 1:1 (for example 100 grams of drying agent per 100 grams of ruthenium precursor) to about 1:100 (for example, 1 gram of drying agent per 100 grams of ruthenium precursor), preferably from about 1:10 (for example 1 gram of drying agent per 10 grams of ruthenium precursor) to about 1:50 (for example, 1 gram of drying agent per 50 grams of ruthenium precursor). In addition, the contact between the ruthenium precursor and the one or more drying agents is carried out under a blanket of inert gas as described hereinbefore, preferably a blanket of nitrogen. The purified ruthenium precursor, once obtained, will also be stored under a blanket of inert gas until used.

Accordingly, in this second embodiment, the process may be carried out on a continuous basis or a batchwise basis. Typically when the batch is small, the process will be a batchwise process while when the batch is large the process will be either batchwise or continuous with the terms “small” and “large” being as defined hereinbefore. In those embodiments where it is desirable to have a large batch continuous processing cycle, it is possible to utilize more than one column thereby allowing for the ruthenium precursor to be run through one column and the succeeding columns as necessary to remove the impurities present. By having these columns is succession, this also allows for one or more of the columns to be taken off line for the regeneration or replacement of drying agent when necessary.

The temperature at which contact in the process of the second embodiment is carried out is also not necessarily critical to the process. Typically, the process will be carried out at about room temperature (25° C.) although higher and lower temperatures are contemplated to be within the scope of the present invention. The lower limit of the temperatures will be determined based on the freezing point of the actual organic solvent and ruthenium precursor utilized. In many instances, those of ordinary skill in the art will recognize that taking into account the freezing point of the organic solvent and ruthenium precursor that the lower limit will typically be no lower than about −20° C. With regard to the upper temperature limit for carrying out this embodiment of the process of the present invention, this limit is determined by the stability of the ruthenium precursor and the organic solvent. Accordingly, the upper limit will typically be at most about 80° C. As noted though, the preferred temperature will be room temperature (25° C.) plus or minus 10° C. (from about 15° C. to about 30° C.).

With regard to this second embodiment, the ratio of drying agent (cumulative amount of drying agent) to ruthenium precursor will typically range from about 1:1 (for example 100 grams of drying agent per 100 grams of ruthenium precursor) to about 1:20 (for example, 5 grams of drying agent per 100 grams of ruthenium precursor), preferably from about 1:2 (for example 50 grams of drying agent per 100 grams of ruthenium precursor) to about 1:10 (for example, 10 grams of drying agent per 100 grams of ruthenium precursor).

With regard to the above parameters, the purification (drying) efficiency will depend upon the impurity level, the freshness of the molecular sieve (whether it is loaded already), the ratio of drying agent to ruthenium precursor, the temperature at which the process is carried out, the contact time, as well as other conditions for processing.

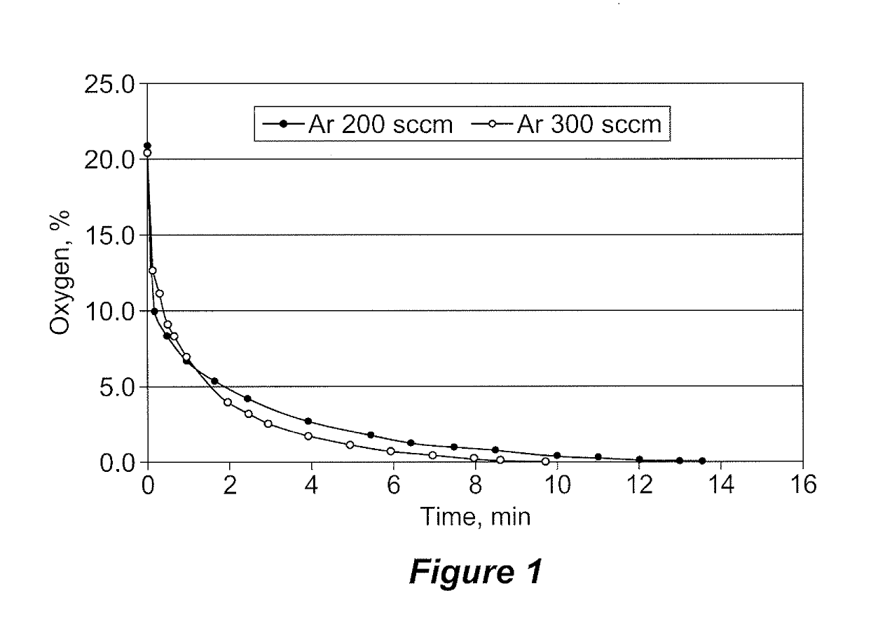

While the synthesis of the raw ruthenium compound can be carried in an air environment for convenient operation, the air, as well as moisture and other impurities, left in the raw product will likely affect film deposition processes since a reducing gas such as hydrogen will have to be used. The impurity gas is likely to cause process instability as well. In order to circumvent this problem, an inert gas may be flowed through the product for a period of time thereby allowing for the oxygen, together with other air gases, to be purged away from the solution. This may be done prior to or after the purification process of the present invention. FIG. 1 provides the oxygen level as a function of purge time and gas flow rate.

After the above purification, the film deposition process is significantly improved. Without purification, film resistance from the raw product may go up to 500 ohm/square. After purification, the film resistance drops to around 15 ohm/square. The film uniformity is improved and batch-to-batch deposition process is more repeatable and consistent.

### EXAMPLES

### Example 1

A ruthenium aqueous phase was prepared by adding cerium ammonium nitrate to de-ionized water and then adding ruthenium nitrosyl aqueous solution. A clear yellow solution was formed. To this solution a fluorinated solvent mixture comprising Ethyl Nonafluorobutyl Ether and Methyl Nonafluorobutyl Ether was added to the aqueous phase and the mixture was stirred. After a period of about five (5) hours, the mixing was stopped and the mixture was allowed to settle. Two separate phases were automatically formed because of the immiscibility. The aqueous phase was removed using a separation funnel and the organic phase which comprises the ruthenium product (ruthenium tetraoxide) and the organic solvent was retained as the raw ruthenium precursor.

The ruthenium precursor that comprises the ruthenium tetraoxide dissolved in a fluorinated solvent mixture comprising ethyl nonafluorobutyl ether and methyl nonafluorobutyl ether was mixed with a freshly activated 4 A molecular sieve and stored for approximately twelve (12) hours in a sealed container under a blanket of dry air. The ruthenium precursor was then placed in a moisture-free gravity type drying and filtration system in a nitrogen environment to remove moisture and particles. The concentration of ruthenium tetraoxide was monitored before and after the drying and filtering processes. No change in the concentration was found.

The dried and filtered product was stored in a container topped with nitrogen gas until ready for use.

### Example 2

A sample of the same ruthenium precursor as used in Example 1 was dried using a column containing activated 4 A molecular sieve in the form of ⅛ inch (0.3 cm) beads. The ruthenium precursor was forced to flow through the column containing the molecular sieve and through an attached particular filter using pressurized Argon gas. Moisture and residual particles were removed and separated from the ruthenium precursor. In this case, drying and filtration were performed in the same system which comprised two separate units that were joined together. The dried and filtered product was cycled through the column and filter again using the same process. The concentration of ruthenium tetraoxide was monitored before and after the drying and filtering processes. No change in the concentration was found. The dried and filtrated product was stored in a container topped with argon gas until ready for use.

What is claimed is:

1. A method of purifying a ruthenium containing precursor, the method comprising:
removing oxygen from the ruthenium containing precursor by flowing an inert gas through the ruthenium containing precursor.

2. The method of claim 1, wherein the ruthenium containing precursor is ruthenium tetraoxide in an organic solvent.

3. The method of claim 2, wherein the inert gas is argon.

4. The method of claim 3, wherein the inert gas is flowed through the ruthenium containing precursor at a flow rate of 200 sccm and for a duration of less than 14 minutes.

5. The method of claim 3, wherein the inert gas is flowed through the ruthenium containing precursor at a flow rate of 300 sccm and for a duration of less than 10 minutes.

6. The method of claim 3, wherein a ruthenium containing film formed by the ruthenium containing precursor has a resistance of approximately 15 ohm/square.

7. The method of claim 3, wherein the method produces a ruthenium containing precursor having an oxygen concentration below approximately 2%.

8. The method of claim 3, wherein the method produces a ruthenium containing precursor having an oxygen concentration below approximately 1%.

9. A method of forming an improved ruthenium containing film, the method comprising:
depositing a ruthenium containing film by using a ruthenium containing precursor having reduced oxygen concentration in CVD, ALD, or PVD.

10. The method of claim 9, wherein the ruthenium containing precursor having reduced oxygen concentration is produced by flowing an inert gas through the ruthenium containing precursor.

11. The method of claim 10, wherein the ruthenium containing precursor is ruthenium tetraoxide in an organic solvent.

12. The method of claim 11, wherein the inert gas is argon.

13. The method of claim 12, wherein the inert gas is flowed through the ruthenium containing precursor at a flow rate of 200 sccm and for a duration of less than 14 minutes.

14. The method of claim 12, wherein the inert gas is flowed through the ruthenium containing precursor at a flow rate of 300 sccm and for a duration of less than 10 minutes.

15. The method of claim 12, wherein the ruthenium containing film has a resistance of approximately 15 ohm/square.

16. The method of claim 12, wherein the oxygen concentration is below approximately 2%.

17. The method of claim 12, wherein the oxygen concentration is below approximately 1%.
